(19) United States
(12) Patent Application Publication
(10) Pub. No.: US 2026/0334790 A1
(43) Pub. Date: Oct. 29, 2026

(54) SYSTEM AND METHOD FOR BIOLOGICAL SIGNAL MEASUREMENT

(71) Applicant: Taiwan-Asia Semiconductor Corporation, Hsinchu City (TW)

(72) Inventors: Kai-Hsiang Su, Hsinchu City (TW); Chia-Te Li, Hsinchu City (TW)

(21) Appl. No.: 19/297,405

(22) Filed: Aug. 12, 2025

(30) Foreign Application Priority Data
Apr. 24, 2025 (TW) 114115600

(51) Int. Cl. A61B 5/00

(52) U.S. Cl. CPC A61B 5/0059

(57) ABSTRACT

A system and method for biological signal measurement are provided. The biological signal measurement system includes an optical module, a thermal sensing module, and a signal processing module. The optical module includes a light-emitting diode and a photodiode, wherein a light emitted by the light-emitting diode is diffusely reflected by a testee and then received by the photodiode to generate a plurality of optoelectronic signals, which vary with temperature changes. The thermal sensing module, disposed adjacent to the light-emitting diode, senses an ambient temperature of the light-emitting diode to generate a plurality of temperature signals. The signal processing module receives the temperature signals and the optoelectronic signals to generate a plurality of data windows, corrects each data window to produce a plurality of compensated data windows, and merges the compensated data windows to generate a clean signal uncorrelated with the ambient temperature, corresponding to a biological signal of the testee.

### CROSS-REFERENCES TO RELATED APPLICATIONS

This application claims the benefit of priority to Taiwanese Patent Application No. 114115600 filed on Apr. 24, 2025, which is hereby incorporated by reference in its entirety.

### BACKGROUND OF THE INVENTION

### Field of the Invention

The present invention relates to a system and method for biological signal measurement, and more particularly to a system and method for biological signal measurement that eliminates thermal noise.

### Descriptions of the Related Art

With the rapid advancement of electronic technology miniaturization, wearable devices equipped with built-in sensors for monitoring human physiological parameters have become increasingly popular, with applications in health monitoring, sports tracking, medical diagnostics, and other fields.

For example, non-invasive blood glucose monitoring technology measures blood glucose levels without the need for skin punctures or blood sample extraction. The most significant advantage of this technology is that it eliminates the need for needles, making it more comfortable than traditional blood glucose testing methods and reducing pain and discomfort for patients. Additionally, non-invasive methods reduce the risk of infection for testees, contributing to improved public health. Moreover, such devices are easy to operate, allowing users to perform measurements at home or in daily life without requiring specialized training or assistance from medical professionals. Most importantly, non-invasive testing enables continuous monitoring, providing detailed, real-time data that facilitates better disease management for patients.

However, non-invasive testing technologies still have certain limitations. For instance, the accuracy of current non-invasive blood glucose testing technologies is relatively low and is affected by various factors, one of which is the impact of ambient temperature on the optical module in wearable devices. Changes in ambient temperature cause variations in the light emission efficiency of the light-emitting diode in the optical module, which indirectly affects the intensity of the signals sensed by the photodiode in the optical module. These temperature-induced variations act as noise in the physiological signals of the testee, leading to system misjudgments in subsequent applications and causing frequent doubts about the accuracy of non-invasive blood glucose testing technologies. To address these issues, there is an urgent need in the industry for an innovative biological signal measurement system and method to improve the aforementioned problems.

### SUMMARY OF THE INVENTION

The primary objective of the present invention is to provide an innovative biological signal measurement system and method that addresses the issues of traditional biological signal measurement systems, which are susceptible to interference from ambient thermal noise and affect measurement accuracy.

To achieve the above objective, the present invention provides a biological signal measurement system comprising an optical module, a thermal sensing module, and a signal processing module. The optical module includes a light-emitting diode and a photodiode, wherein a light emitted by the light-emitting diode is diffusely reflected by a testee and received by the photodiode to generate a plurality of optoelectronic signals, which vary with temperature changes. The thermal sensing module is disposed adjacent to the light-emitting diode to sense an ambient temperature of the light-emitting diode and generate a plurality of temperature signals. The signal processing module receives the temperature signals and the optoelectronic signals to generate a plurality of data windows, corrects each data window to produce a plurality of compensated data windows, and merges the compensated data windows to generate a clean signal uncorrelated with the ambient temperature, corresponding to a biological signal of the testee.

In one embodiment of the biological signal measurement system of the present invention, the data windows include an initial reference dataset and a plurality of continuous data windows, and the signal processing module uses a temperature compensation calculation model to correct each continuous data window corresponding to the initial reference dataset and generate each compensated data window.

In one embodiment of the biological signal measurement system of the present invention, the signal processing module employs an artifact subspace reconstruction (ASR) method as the temperature compensation calculation model, wherein the temperature compensation calculation model establishes and identifies a non-temperature component subspace and a temperature noise subspace based on each data window, removes the temperature noise subspace while retaining only the non-temperature component subspace, and reconstructs the signal from the non-temperature component subspace to form each compensated data window.

In one embodiment of the biological signal measurement system of the present invention, the signal processing module performs signal preprocessing after receiving the temperature signals and the optoelectronic signals, wherein the signal preprocessing includes a median filter processing, a moving average processing, and a standardization processing.

In one embodiment of the biological signal measurement system of the present invention, the system further includes a substrate, wherein the optical module and the thermal sensing module are disposed on the substrate.

To achieve the above objective, the present invention provides a biological signal measurement method comprising the following steps: providing a light-emitting diode to emit a light to a testee; providing a photodiode to receive the light diffusely reflected by the testee to generate a plurality of optoelectronic signals, wherein the optoelectronic signals vary with temperature changes; providing a thermal sensing module disposed adjacent to the light-emitting diode to sense an ambient temperature of the light-emitting diode and generate a plurality of temperature signals; providing a signal processing module to receive the temperature signals and the optoelectronic signals to generate a plurality of data windows; correcting each data window to produce a plurality of compensated data windows; and merging the compensated data windows to generate a clean signal uncorrelated with the ambient temperature, corresponding to a biological signal of the testee.

In one embodiment of the biological signal measurement method of the present invention, the data windows include an initial reference dataset and a plurality of continuous data windows, and the signal processing module uses a temperature compensation calculation model to correct each continuous data window corresponding to the initial reference dataset and generate each compensated data window.

In one embodiment of the biological signal measurement method of the present invention, the signal processing module employs an artifact subspace reconstruction (ASR) method as the temperature compensation calculation model, wherein the temperature compensation calculation model establishes and identifies a non-temperature component subspace and a temperature noise subspace based on each data window, removes the temperature noise subspace while retaining only the non-temperature component subspace, and reconstructs the signal from the non-temperature component subspace to form each compensated data window.

In one embodiment of the biological signal measurement method of the present invention, the temperature compensation calculation model uses principal component analysis (PCA) on each data window to establish and identify the non-temperature component subspace and the temperature noise subspace.

In one embodiment of the biological signal measurement method of the present invention, the step of providing a signal processing module further includes a step of signal preprocessing for performing a median filter processing, a moving average processing, and a standardization processing after receiving the temperature signals and the optoelectronic signals by the signal processing module.

After referring to the drawings and the embodiments as described in the following, those the ordinary skilled in this art can understand other objectives of the present invention, as well as the technical means and embodiments of the present invention.

### BRIEF DESCRIPTION OF THE DRAWINGS

FIG. 1 is a conceptual schematic diagram illustrating the measurement of a testee's biological signal using the biological signal measurement system of the present invention;

FIG. 2 is a schematic diagram illustrating the signal processing for calibrating optoelectronic signals using temperature signals in the biological signal measurement system of the present invention;

FIG. 3 is a schematic diagram illustrating the operational flow of the biological signal measurement system of the present invention;

FIG. 4A to FIG. 4J are schematic diagrams illustrating temperature compensation correction in one embodiment of the biological signal measurement system of the present invention; and

FIG. 5 is a flowchart illustrating the biological signal measurement method of the present invention.

### DETAILED DESCRIPTION OF THE PREFERRED EMBODIMENT

In the following description, the present invention will be explained with reference to various embodiments thereof. These embodiments of the present invention are not intended to limit the present invention to any specific environment, application or particular method for implementations described in these embodiments. Therefore, the description of these embodiments is for illustrative purposes only and is not intended to limit the present invention. It shall be appreciated that, in the following embodiments and the attached drawings, a part of elements not directly related to the present invention may be omitted from the illustration, and dimensional proportions among individual elements and the numbers of each element in the accompanying drawings are provided only for ease of understanding but not to limit the present invention.

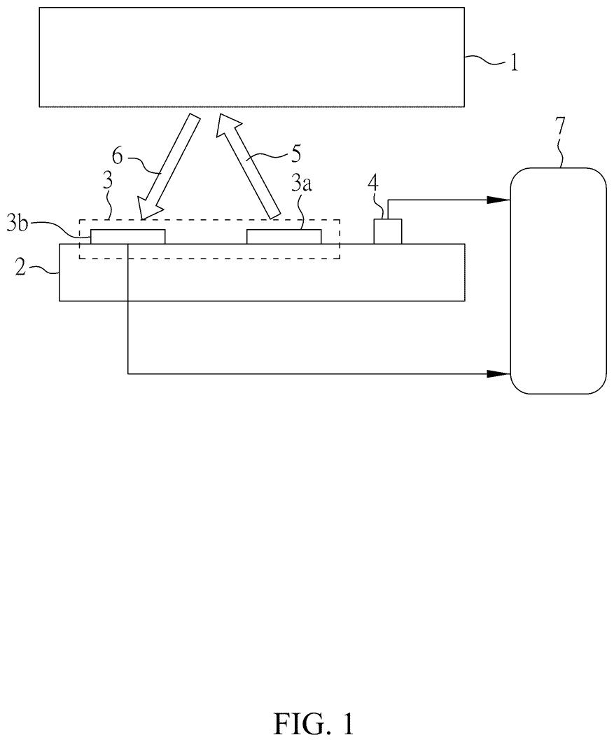

Referring to FIG. 1, a conceptual schematic diagram illustrating the measurement of a testee's biological signal using the biological signal measurement system of the present invention is shown. Specifically, the biological signal measurement system of the present invention is a miniaturized measurement module applied in a wearable device to measure physiological signals of a testee 1. The system includes a substrate 2, an optical module 3, and a thermal sensing module 4. The optical module 3 and the thermal sensing module 4 are both disposed on the substrate 2 and embedded in the wearable device. The optical module 3 includes a light-emitting diode 3a and a photodiode 3b, wherein the light-emitting diode 3a emits light in the wavelength range of approximately 700 nanometers (nm) to 1700 nanometers (nm), falling within the near-infrared (NIR) and/or short-wave infrared (SWIR) spectrum. The photodiode 3b receives external light and generates a corresponding photocurrent signal. In an application of this embodiment, the light 5 emitted by the light-emitting diode 3a enters the epidermal tissue of the testee 1, interacts with different tissue substances inside the testee's body (e.g., water, glucose, fat, hemoglobin, and proteins) through scattering and absorption, and is diffusely reflected as light 6, which exits the testee's body and is received by the photodiode 3b and a plurality of optoelectronic signals are generated and used for subsequent biological signal analysis.

It should be noted that the intensity of the light 5 emitted by the light-emitting diode 3a during operation is directly affected by the ambient environment or the heat generated by the module itself, causing variations with temperature changes. These variations also affect the intensity of the diffusely reflected light 6 from the testee's epidermal tissue, which indirectly influences the strength of the optoelectronic signals generated by the photodiode 3b. Such temperature-induced variations act as noises in the measurement of specific tissue substances in the testee's body, significantly impacting the accuracy of biological signal analysis. Therefore, the present invention specifically arranges a thermal sensing module 4 to record the temperature signals of the light-emitting diode 3a for correcting the raw measurement values of the photodiode 3b corresponding to the temperature's effect on the light-emitting diode's emission efficiency, thereby eliminating system misjudgments caused by thermal noises.

Additionally, the biological signal measurement system of the present invention further includes a signal processing module 7 that utilizes a temperature compensation calculation model for system temperature compensation. Specifically, the signal processing module 7 receives temperature signals recorded by the light-emitting diode 3a and raw optoelectronic signals measured by the photodiode 3b, corrects the measured values corresponding to the temperature's effect on the light-emitting diode's emission efficiency, eliminates the temperature's impact on the optoelectronic signal measurements, and ensures that the sensed signals from the photodiode in the wearable device correspond only to changes in specific tissue substances in the testee's body for better determining variation trends thereof.

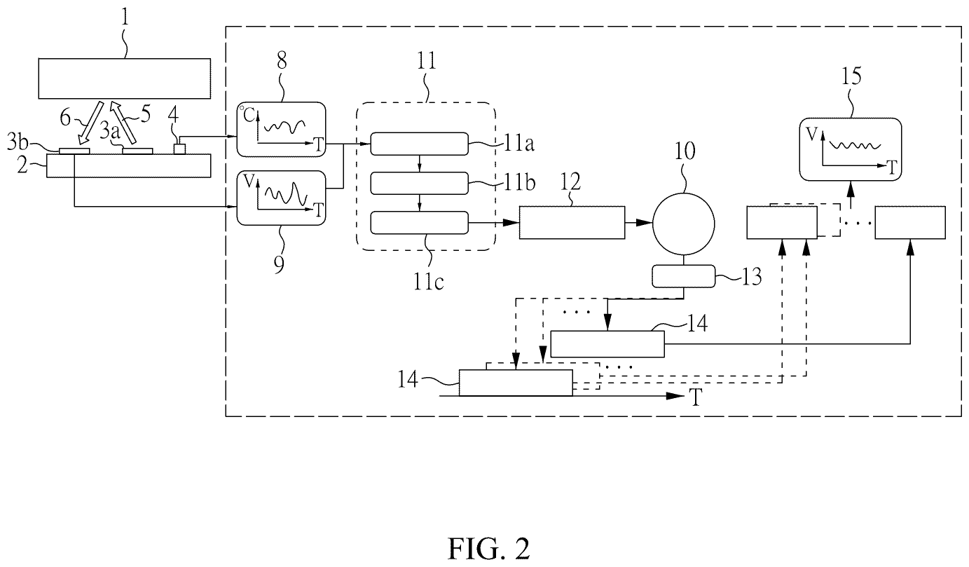

Referring to FIG. 2, a schematic diagram illustrates the signal processing for calibrating optoelectronic signals using temperature signals in the biological signal measurement system of the present invention. Preferably, the thermal sensing module 4 is positioned as close as possible to the light-emitting diode 3a to accurately detect the ambient temperature near the light-emitting diode for facilitating subsequent elimination of thermal noise in the system. Furthermore, the biological signal measurement system of the present invention is a real-time updating system, ensuring that signals corrected and compensated by the system are unaffected by temperature fluctuations during specific time periods, accurately presenting the biological signals of the testee's body. Specifically, the biological signal measurement system of the present invention continuously receives temperature signals 8 from the light-emitting diode 3a and optoelectronic signals 9 from the photodiode 3b, continuously calibrates the optoelectronic signals 9 using the temperature compensation calculation model 10, updates the calculation model of the system, and outputs testee's accurate biological signals after obtaining the optoelectronic signals unaffected by temperature variations.

It should be noted that, after the wearable device employing the present invention begins operation, the signal processing module 7 continuously receives dynamically changing temperature signals 8 and optoelectronic signals 9, sequentially filling these signals into a data buffer. Once a data window is formed (for example, each data window containing 15 data points), signal preprocessing 11 is performed on the formed data window to filter out unreasonably strong or weak noise that may occur during the measurement process and to standardize the temperature signals 8 and optoelectronic signals 9 to ensure comparability and operability.

For example, as shown in FIG. 2, signal preprocessing 11 may include median filter processing 11a, moving average processing 11b, and standardization processing 11c. The median filter processing 11a filters out abnormally intense outlier signals from the signals. That is to select several data points based on the time sequence and filter them backward (for example, selecting five signals forward from the current point, sliding the data window backward by one data point with inputting each new data to replace one existed signal while maintaining the total length of the data window), taking the median of the sampled data points as the signal intensity for that time point, and repeating this process until the entire signal length of the data window is updated, thereby eliminating abnormally high or low outlier signals. The moving average processing 11b involves continuously updating rolling data corresponding to the time sequence. Similar to the median filter process, it calculates the average of a fixed number of data points and repeats until the entire signal length is updated for further smoothing the received signals. These steps are repeated until a complete data window is filled with preprocessed signals. Then, the standardization processing 11c is followed to make the two types of data with different physical quantities comparable and operable for the next stage of temperature compensation processing. After the standardization processing 11c, the data window is transmitted to the temperature compensation calculation model 10 for temperature correction and compensation processing. It is noted that parameters such as the mean and standard deviation of the preprocessed data are retained in the system for use in restoring the original physical quantities of the signals after temperature correction.

Continuing to refer to FIG. 2, when the wearable device equipped with the measurement system of the present invention operates, the photodiode 3b continuously receives diffusely reflected light 6 containing the testee's biological signals, which also includes thermal noises. To address the need for temperature calibration, the biological signal measurement system of the present invention establishes a temperature compensation mechanism to remove thermal noises. Given the continuous physiological monitoring capability of the system, the signal processing module 7 utilizes the flow of continuous data, including continuous temperature signals 8 and optoelectronic signals 9, to create multiple “sliding” data windows 12. The temperature compensation calculation model 10 performs real-time temperature compensation correction 13 on the data windows 12 to generate compensated data windows 14, which are stored in a data buffer. Finally, the compensated data windows 14 in the data buffer are merged to generate a clean signal 15 uncorrelated with the ambient temperature, corresponding to a biological signal of the testee 1, facilitating subsequent physiological feature transformation and analysis.

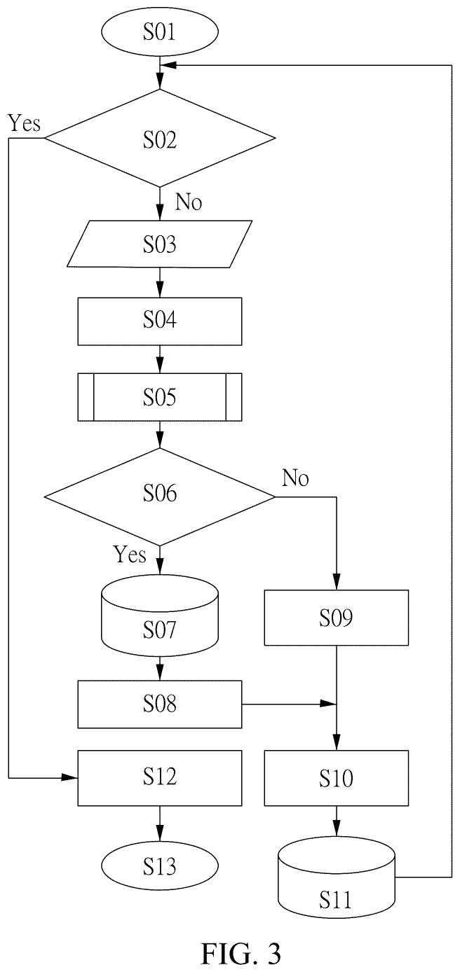

Referring to FIG. 2 and FIG. 3 together, FIG. 3 illustrates a schematic diagram of the operational flow of the biological signal measurement system of the present invention. Specifically, in step S01, when the biological signal measurement system begins operation, it starts and continuously collects new data points. In step S02, the system determines whether all data points have been collected. If data collection is incomplete, the process proceeds to step S03, where additional data points, including temperature signals 8 from the light-emitting diode 3a and optoelectronic signals 9 from the photodiode 3b, are collected. In step S04, after a certain period, these data points accumulate until a complete data window length is achieved, forming a data window. In step S05, signal preprocessing 11 is performed on each data window to eliminate extreme values and smooth and standardize the signals within the data window. As data points are added, the data window continuously “slides” backward until a complete data window 12 of preprocessed signals is filled.

Continuing to refer to FIG. 2 and FIG. 3, in step S06, the system determines whether the data window is the first data window of the biological signal measurement system. If so, in step S07, the first data window is set as the initial reference signal. Then, in step S08, the initial reference signal is used to calculate and establish the temperature compensation calculation model 10, which performs temperature compensation correction 13 on the second and subsequent data windows. Specifically, in step S09, when the system determines that the subsequent input data window is the second data window, the initial reference signal is incorporated, and in step S10, the temperature compensation calculation model 10 is used to perform calculations for temperature compensation correction 13 on the second data window to remove its temperature components. In step S11, a compensated data window 14, free of temperature components, is generated. Similarly, subsequent data windows (third, fourth, etc.) repeat steps S09 to S11 to remove temperature components. It is noted that the present invention incorporates an adaptive mechanism that adjusts the temperature compensation model and continuously updates the reference data to adapt to the temperature variation patterns of the entire dataset. Specifically, each compensated data window 14 generated after temperature compensation correction is sequentially added to a reference data buffer in the signal processing module 7, where previously completed reference data is used for subsequent calculations for temperature compensation correction 13 using the temperature compensation calculation model 10. Concurrently, the compensated data windows 14 are also added to a buffer for corrected windows. When the system determines in step S02 that data collection is complete, step S12 is executed, merging all compensated data windows 14 in the corrected window buffer into complete temperature-compensated data to generate a clean signal 15 uncorrelated with the ambient temperature. In step S13, the compensation correction calculation is completed after outputting the clean signal 15.

It should be noted that the signal processing module of the biological signal measurement system of the present invention employs an Artifact Subspace Reconstruction (ASR) method as the temperature compensation calculation model, which removes noise components from the feature subspace of signals in each data window, retaining only the clean reference signal components, such as rejecting or removing the temperature components of the reference signal. Specifically, the calculation process first derives a mixing matrix from the covariance matrix of the reference data, then uses Principal Component Analysis (PCA) to establish and identify a temperature noise subspace and a non-temperature component subspace. The temperature noise subspace is removed, retaining only the non-temperature component subspace, which is then projected onto the mixing matrix to reconstruct the corrected signal. The calculation process is as follows:
1. Decompose the new data window Xₜ into a mixing matrix Mᵣ from the reference signal and a source signal Sₜ containing temperature noises:

Xₜ=MᵣSₜ (Equation 1)
2. Perform PCA to Xₜ by using the reference signal to obtain the principal component representation Yₜ and source signal Sₜ:

$${Y}_{t} = {V}_{t}^{T} {X}_{t} = {t}_{r}^{T} {M}_{r} {S}_{t} \qquad ( Equation 2 )$$

$${S}_{t} = {( {V}_{t}^{T} {M}_{r} )}^{T} {Y}_{t} \qquad ( Equation 3 )$$

where Vₜ is the eigenvector of the covariance matrix of the reference signal.
3. Reject the temperature components in Yₜ for obtaining the corrected source signal (Sₜ) correct:

$${( {S}_{t} )}_{correct} = {( {V}_{t}^{T} {M}_{r} )}_{trunc}^{+} {Y}_{t} \qquad ( Equation 4 )$$

where trunc indicates zeroing the temperature subspace, and superscript ⁺ indicates the pseudoinverse matrix.
4. Finally, project back to Mᵣ to reconstruct the compensated data window (Xₜ)_{correct}:

$${( {X}_{t} )}_{correct} = {{M}_{r} ( {V}_{t}^{T} {M}_{r} )}_{trunc}^{+} {V}_{t}^{T} {X}_{t} \qquad ( Equation 5 )$$

where subscript t indicates the new window data, and subscript r indicates the reference signal.

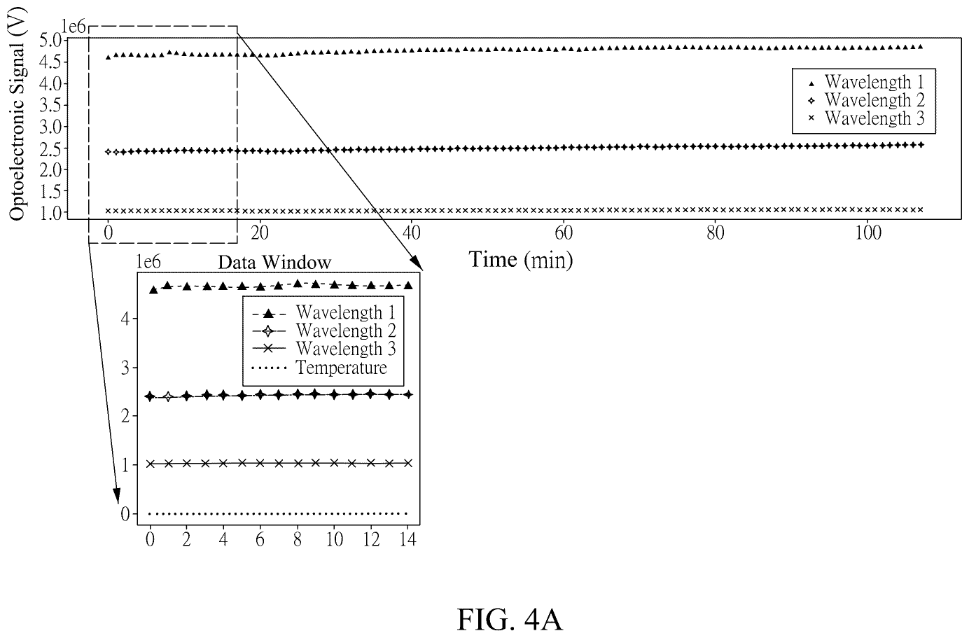

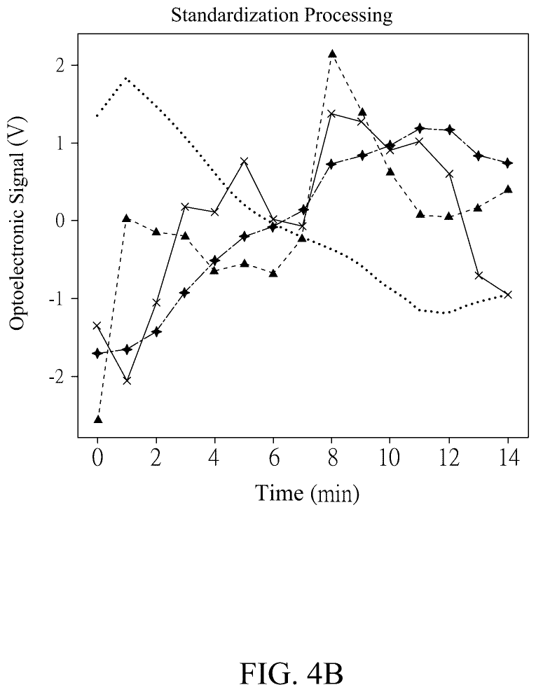

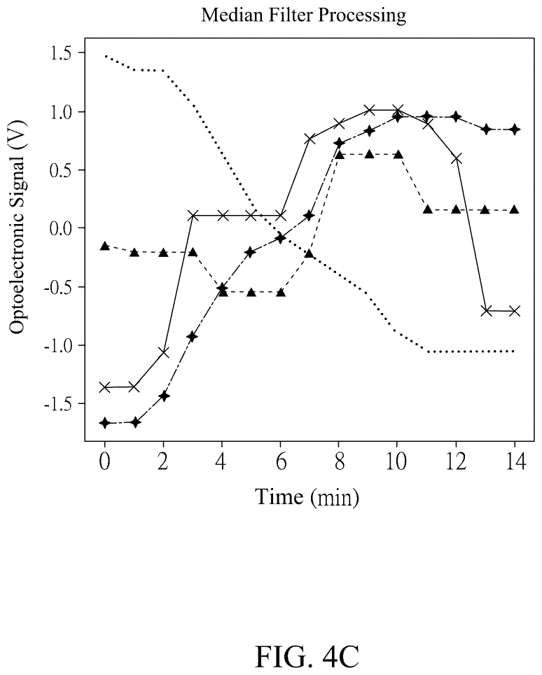

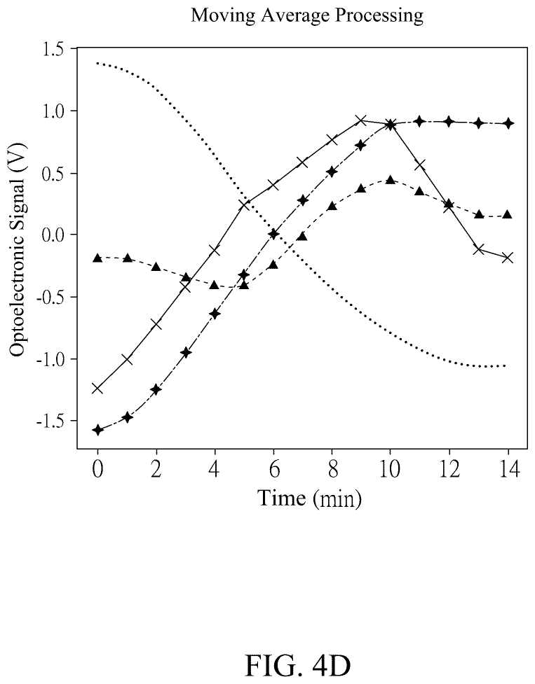

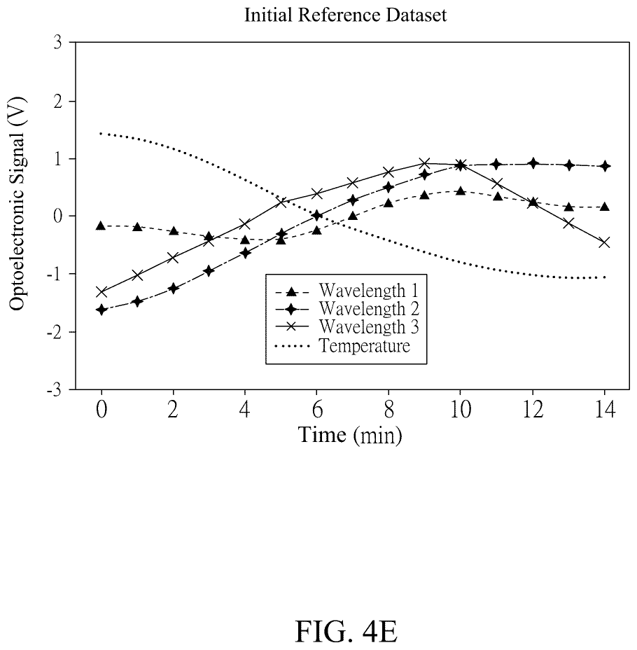

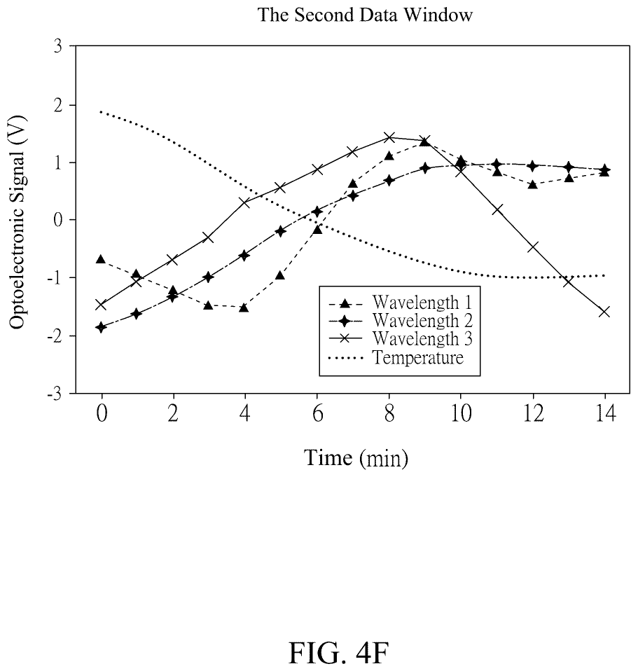

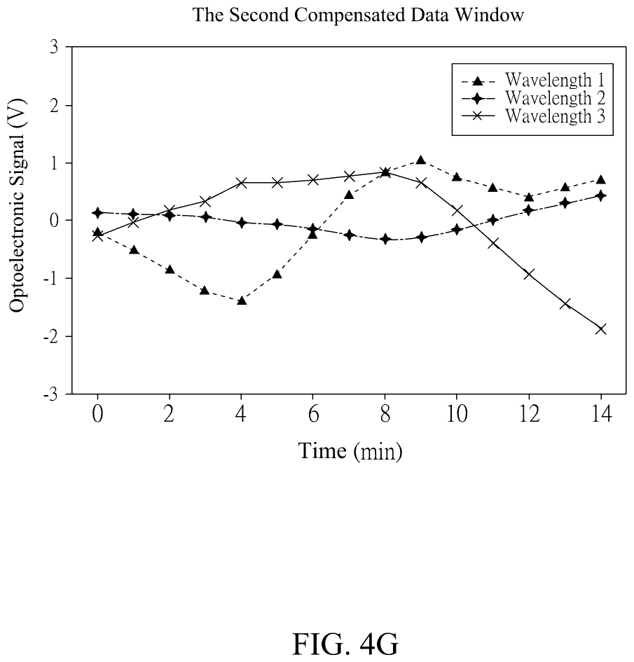

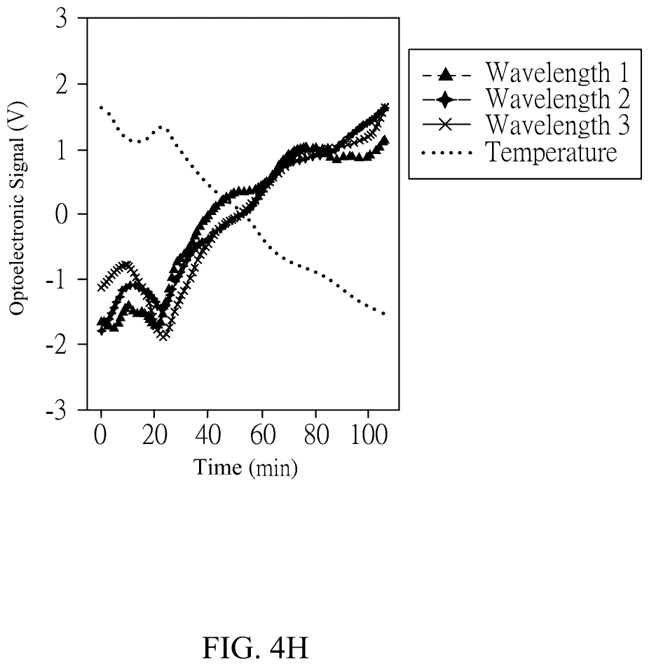

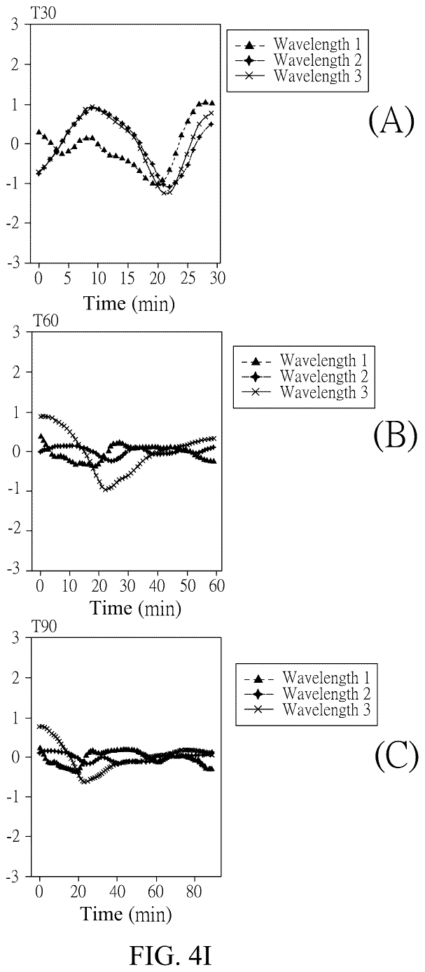

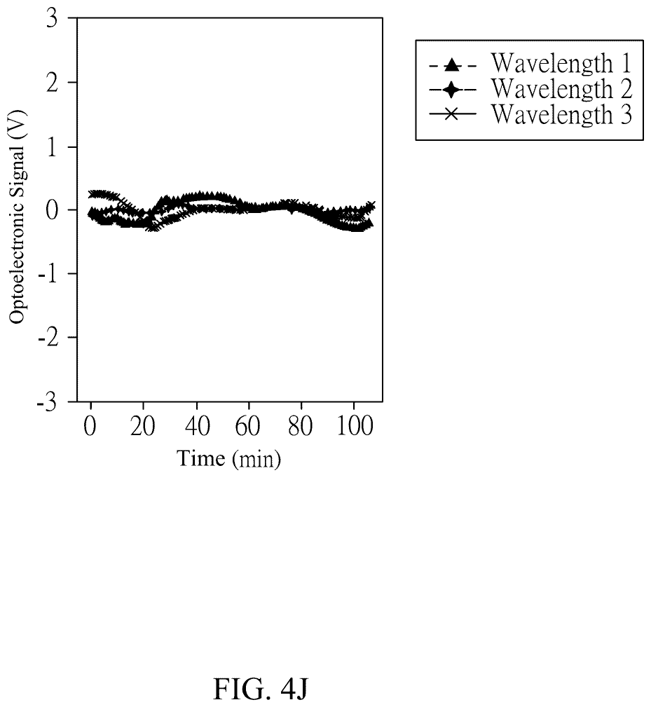

The following embodiment illustrates how the biological signal measurement system of the present invention removes temperature components from optoelectronic signals to achieve accurate biological signal measurement. Referring to FIG. 4A to FIG. 4J, FIG. 4A shows a complete dataset demonstrating the temperature compensation mechanism of the present invention, with three optoelectronic signals and one temperature signal simulating real-time temperature correction. Additionally, FIG. 4A shows a selection of 15 data points as a single data window, designated as the first data window. As shown in FIG. 4B, the first data window undergoes standardization processing to ensure subsequent data processability. Next, signal preprocessing is performed on the first data window, including median filter processing as shown in FIG. 4C and moving average processing as shown in FIG. 4D, to remove abnormal outliers and smooth the signal. The preprocessed first data window is set as initial reference dataset of the system, as shown in FIG. 4E.

In this embodiment, one data point is set as a sliding length of the data window per each slide. That is, when a new data is received, the data widow will slide backward one data point for maintaining a constant total data window length. As shown in FIG. 4F, the second data window is processed using the initial reference dataset and the temperature compensation calculation model for generating the results corrected by temperature compensation shown in FIG. 4G. Compared to the uncorrected new window in FIG. 4F, the signals in FIG. 4G show that temperature components have been eliminated in the three optoelectronic signals, with temperature-induced offsets compensated. The corrected signals accurately correspond to a biological signal of the testee, such as blood glucose trends.

Referring to FIG. 4H to FIG. 4J, FIG. 4H shows the complete standardized data without temperature correction, where the three optoelectronic signal curves and the temperature curve exhibit clear opposing trends. In contrast, FIG. 4I includes three curves (A), (B), and (C), representing the results of temperature compensation correction at the 30th, 60th, and 90th data points, respectively, using the biological signal measurement method of the present invention. Additionally, compared to FIG. 4H, FIG. 4J shows the results of offline temperature compensation correction for the data in FIG. 4H. Comparing FIG. 4H to FIG. 4J, it is evident that the more data points the measurement system includes, the closer the temperature compensation correction results are to the offline correction control group, effectively eliminating temperature noises to the true signals.

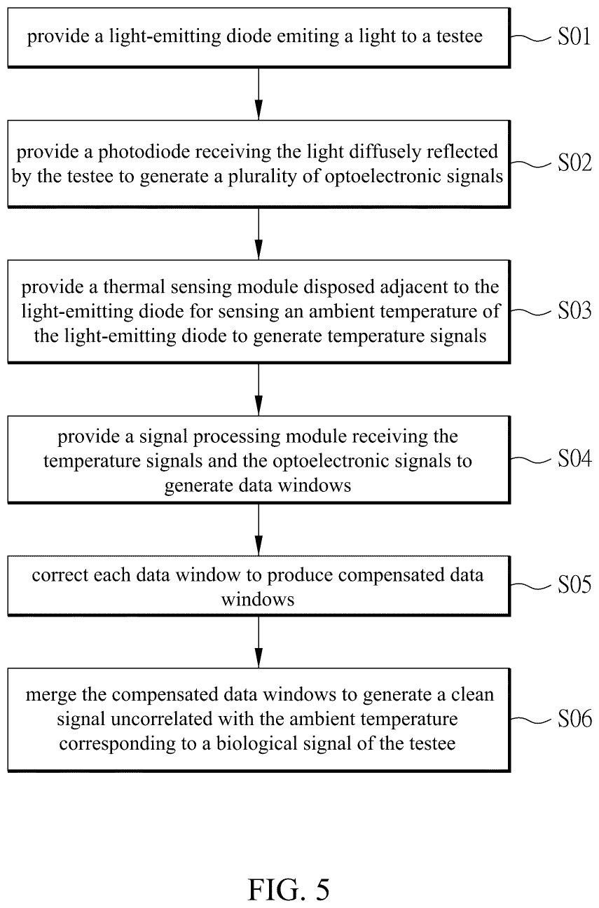

Referring to FIG. 5, a flowchart illustrates the biological signal measurement method of the present invention. In step S01, provide a light-emitting diode emitting a light to a testee. In step S02, provide a photodiode receiving the light diffusely reflected by the testee to generate a plurality of optoelectronic signals, which vary with temperature changes. In step S03, provide a thermal sensing module disposed adjacent to the light-emitting diode for sensing an ambient temperature of the light-emitting diode to generate a plurality of temperature signals. In step S04, provide a signal processing module receiving the temperature signals and the optoelectronic signals to generate a plurality of data windows. In step S05, correct each data window to produce a plurality of compensated data windows. In step S06, merge the compensated data windows to generate a clean signal uncorrelated with the ambient temperature corresponding to a biological signal of the testee. Detailed descriptions of related components can refer to the above content and are not repeated here.

The above embodiments are provided to illustrate the implementations of the present invention and to explain its technical features, and are not intended to limit the scope of the present invention. Any modifications or equivalent arrangements that can be easily accomplished by those skilled in the art fall within the scope of the present invention, and the scope of the present invention should be defined by the claims.

What is claimed is:

1. A biological signal measurement system, comprising:
an optical module including a light-emitting diode and a photodiode, wherein a light emitted by the light-emitting diode is diffusely reflected by a testee and received by the photodiode to generate a plurality of optoelectronic signals, wherein the optoelectronic signals vary with temperature changes;
a thermal sensing module disposed adjacent to the light-emitting diode to sense an ambient temperature of the light-emitting diode and generate a plurality of temperature signals; and
a signal processing module, after receiving the temperature signals and the optoelectronic signals to generate a plurality of data windows, correcting each of the data windows to produce a plurality of compensated data windows, and merging the compensated data windows to generate a clean signal uncorrelated with the ambient temperature, corresponding to a biological signal of the testee.

2. The biological signal measurement system of claim 1, wherein the data windows include an initial reference dataset and a plurality of continuous data windows, and the signal processing module uses a temperature compensation calculation model to correct each of the continuous data windows corresponding to the initial reference dataset and generate each of the compensated data windows.

3. The biological signal measurement system of claim 2, wherein the signal processing module employs an artifact subspace reconstruction (ASR) method as the temperature compensation calculation model, wherein the temperature compensation calculation model establishes and identifies a non-temperature component subspace and a temperature noise subspace based on each of the data windows, removes the temperature noise subspace while retaining only the non-temperature component subspace, and reconstructs the signal from the non-temperature component subspace to form each of the compensated data windows.

4. The biological signal measurement system of claim 1, wherein the signal processing module performs signal preprocessing after receiving each of the temperature signals and each of the optoelectronic signals, wherein the signal preprocessing includes a median filter processing, a moving average processing, and a standardization processing.

5. The biological signal measurement system of claim 1, further comprising a substrate, wherein the optical module and the thermal sensing module are disposed on the substrate.

6. A biological signal measurement method, comprising:
providing a light-emitting diode to emit a light to a testee;
providing a photodiode to receive the light diffusely reflected by the testee to generate a plurality of optoelectronic signals, wherein the optoelectronic signals vary with temperature changes;
providing a thermal sensing module disposed adjacent to the light-emitting diode to sense an ambient temperature of the light-emitting diode and generate a plurality of temperature signals;
providing a signal processing module to receive the temperature signals and the optoelectronic signals to generate a plurality of data windows;
correcting each of the data windows to produce a plurality of compensated data windows; and
merging the compensated data windows to generate a clean signal uncorrelated with the ambient temperature, corresponding to a biological signal of the testee.

7. The biological signal measurement method of claim 6, wherein the data windows include an initial reference dataset and a plurality of continuous data windows, and the signal processing module uses a temperature compensation calculation model dataset to correct each continuous data window corresponding to the initial reference dataset and generate each of the compensated data windows.

8. The biological signal measurement method of claim 7, wherein the signal processing module employs an artifact subspace reconstruction (ASR) method as the temperature compensation calculation model, wherein the temperature compensation calculation model establishes and identifies a non-temperature component subspace and a temperature noise subspace based on each of the data windows, removes the temperature noise subspace while retaining only the non-temperature component subspace, and reconstructs the signal from the non-temperature component subspace to form each of the compensated data windows.

9. The biological signal measurement method of claim 8, wherein the temperature compensation calculation model uses principal component analysis (PCA) on each of the data windows to establish and identify the non-temperature component subspace and the temperature noise subspace.

10. The biological signal measurement method of claim 6, wherein the step of providing a signal processing module further includes a step of signal preprocessing for performing a median filter processing, a moving average processing, and a standardization processing after receiving each of the temperature signals and each of the optoelectronic signals by the signal processing module.
